(19) United States
(12) Patent Application Publication
(10) Pub. No.: US 2009/0275807 A1
(43) Pub. Date: Nov. 5, 2009

(54) METHOD FOR MANAGING ALARMS IN A PHYSIOLOGICAL MONITORING SYSTEM

(75) Inventors: David Alan Sitzman, Hubertus, WI (US); Timothy Lee Johnson, Niskayuna, NY (US); Xi Wang, Niskayuna, NY (US); Stephen Thomas Treacy, Menomonee Falls, WI (US); Sahika Genc, Troy, NY (US)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: GENERAL ELECTRIC COMPANY, SCHENECTADY, NY (US)

(21) Appl. No.: 12/114,119

(22) Filed: May 2, 2008

(51) Int. Cl. A61B 5/00; G08B 23/00

(52) U.S. Cl. 600/301; 340/573.1

(57) ABSTRACT

A method for managing alarm events in a physiological monitoring system is described. The method includes validating the accuracy of alarm events by checking if the alarm events are noise events. The method further includes identifying a pattern in alarm sequence or an alarm rate of at least one alarm type associated with the alarm events. The alarm rate is the frequency of the occurrence of alarm events for the particular alarm type. Based on the identified pattern in the alarm sequence and the alarm rate and patient data, an alarm level associated with the alarm type is adjusted. The hospital staff is notified depending on the criticality of the adjusted alarm level. Further, the alarm signals are suppressed when either a patient intervention or a pause signal is detected by the physiological monitoring system.

### BACKGROUND

The invention relates generally to physiological monitoring systems, and more particularly to managing alarms generated in the physiological monitoring system.

A physiological monitoring system typically includes a plurality of monitoring stations to monitor alarm signals associated with a plurality of patients. Alarm levels are assigned to alarm signals to indicate current physiological condition of the patients. Based on the alarm levels, the physiological monitoring system provides vital information related to the condition of the patients to the hospital staff. Accordingly, the hospital staff provides appropriate service (termed patient intervention) to the patients. Typical physiological monitoring systems notify the hospital staff based on the alarm level associated with a pattern detected in the monitored waveform of the alarm signals. However, inappropriate alarm signals may be generated in the physiological monitoring system when a poor quality waveform is recorded. The inappropriate alarm signals may also be generated due to a patient intervention. Accordingly, a false notification may be provided to the hospital staff to service the patients. This may divert the attention of the hospital staff from seriously ill patients, with a possible reduction in the quality of services provided to these patients. Further, the current physiological monitoring systems do not provide an indication of when or whether the patient has been attended by the hospital staff, or whether the service provided to the patient has been completed.

Known methods for reducing the number of inappropriate alarms include a bedside pause feature that is provided in some monitoring stations. The hospital staff may use the bedside pause feature to silence the alarm signals while providing service to the patients. However, the hospital staff often does not use the pause feature effectively. Another known method includes silencing all the alarm signals in the physiological monitoring system for a short period of time from a central location (e.g., the ward nursing station) when a patient is being serviced. The duration for which all the alarm signals are silenced is defined by the medical equipment certification standards based on the criticality of the patient's condition. However, this method may allow the alarm signals to sound repeatedly (if the underlying problem is not resolved), or at a frequency that is medically inappropriate for some patient conditions. Yet another known method for managing alarm signals includes adjusting the alarm levels from “factory default” settings to accommodate individual patient conditions, or to accommodate ongoing service. However, a majority of hospitals do not manually adjust the alarm levels from the default settings.

Therefore, there exists a need for managing alarms in the physiological monitoring system, which will adapt notification practices in real time to patient service protocols.

### BRIEF DESCRIPTION

In an exemplary embodiment of the invention, a method for managing one or more alarm events in a physiological monitoring system is provided. The method includes validating the accuracy of the one or more alarm events associated with one or more alarm signals received from a patient. The method further includes identifying a pattern in the sequence of the one or more alarm events of at least one alarm type, which is associated with the one or more alarm signals received. An alarm level associated with the at least one alarm type is adjusted based on the identified pattern in the sequence of the alarm events and patient data. The patient data may include at least one of an age of the patient, gender of the patient, historical disease data of the patient, and historical alarm level data associated with the patient. The hospital staff responds to the one or more alarm events based on the adjusted alarm level. The method further includes suppressing the one or more alarm events based on one or more pre-defined conditions. The one or more pre-defined conditions include at least one of patient intervention and activation of a bedside pause signal or centralized alarm silencing. The alarm level is automatically reset, following patient intervention, when the one or more alarm signals are suppressed. Patient intervention is defined herein to be the action of a caregiver, hospital aide, or visiting family member to alleviate any medical or comfort condition of the patient—for instance, suctioning, use of a respirator, adjusting an intravenous tube, moving the pillow, changing bed height, or assisting with bathroom needs. Most of these activities introduce disturbances to the recorded signals causing false alarm patterns.

In another exemplary embodiment of the invention, a physiological monitoring system is provided. The system includes one or more monitoring stations to monitor one or more alarm events associated with one or more alarm signals related to one or more patients. The one or more alarm events are associated with at least one alarm type. The system also includes an alarm level adaptation module that is configured to identify a pattern in the sequence of an alarm event of the at least one alarm type. The alarm level adaptation module is also configured to adjust an alarm level associated with the at least one alarm type based on the identified pattern in the alarm event sequence, an alarm rate and patient data. The alarm level adaptation module is further configured to suppress the one or more alarm events based on one or more pre-defined conditions. The one or more pre-defined conditions include at least one of a patient intervention and a pause signal. The alarm level associated with a pattern is automatically reset after an alarm pattern ceases or after a patient intervention has occurred.

### DRAWINGS

These and other features, aspects, and advantages of the present invention will become better understood when the following detailed description is read with reference to the accompanying drawings in which like characters represent like parts throughout the drawings, wherein:

FIG. 1 illustrates a physiological monitoring system in accordance with an exemplary embodiment of the invention;

FIG. 2 is a flowchart illustrating a method for managing alarm events in a physiological monitoring system in accordance with an exemplary embodiment of the invention;

FIG. 3 is a table illustrating an alarm level management strategy for various alarm types in accordance with an exemplary embodiment of the invention; and

FIG. 4 is a block diagram illustrating a PVC alarm state machine in accordance with an exemplary embodiment of the invention.

### DETAILED DESCRIPTION

Various embodiments of the invention provide a method and system for managing alarm events in a physiological monitoring system particularly a centralized patient monitoring system.

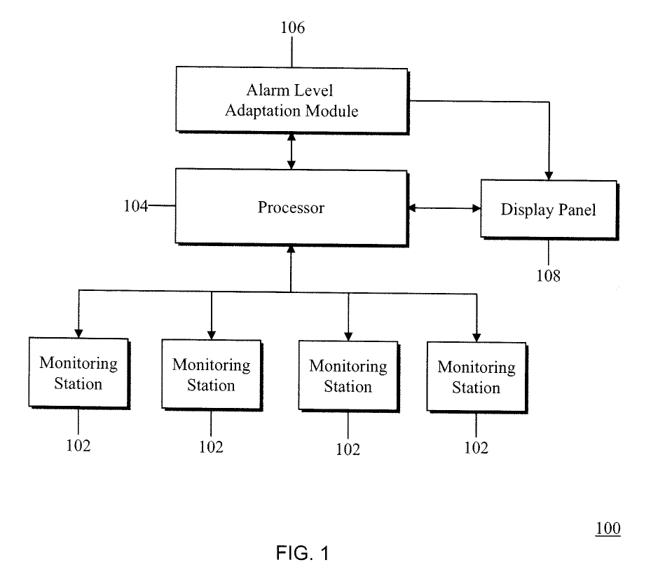

FIG. 1 illustrates a physiological monitoring system 100 in accordance with an exemplary embodiment of the invention. The physiological monitoring system 100 includes one or more monitoring stations 102, a processor 104, an alarm level adaptation module 106, and a display panel 108. The one or more monitoring stations 102 monitor one or more events associated with one or more physiological signals associated with one or more patients in a hospital, and may be either wired (termed a “bedside physiological monitor”) or wireless. The one or more alarm events reflect the physiological condition of the one or more patients. The monitoring stations 102 may monitor, for example, one or more of an electrocardiogram (ECG), non-invasive blood pressure (NBP) and specific blood oxygen (SPO2).

The processor 104 is configured to validate the accuracy of the one or more alarm signals generated by the monitoring stations 102 by checking if the alarm events are noise events, such as an isolated alarm signal, and may perform other tasks such as network management, display generation, and archiving, or interfacing to patient's electronic medical records. Alarm signals are associated with at least one alarm type. When certain features are detected in the alarm signals an alarm event is indicated. Accordingly, the alarm event is also associated with the at least one alarm type. The alarm level adaptation module 106 is configured to set initial alarm levels for each alarm type based on the alarm type and the medical condition of the patient. The alarm level adaptation module 106 is also configured to identify a pattern in the sequence of detected alarm events of one or more alarm type. The pattern in the alarm events is tracked for a pre-defined time interval. The pattern or trend in the detected alarm events may be tracked for example by tracking an alarm rate associated with the alarm type. The alarm rate may be defined as the short-term average frequency of occurrence of an alarm event associated with an alarm type. Examples of the alarm type can be, but are not limited to, Premature Ventricular contraction (PVC), Asystole, Artifact, Heart Rate (HR), Couplet and Tachycardia. Examples of the alarm levels include, but are not limited to, System Message (Level 1), System Advisory (Level 2), System Warning (Level 3), Message (Level 4), Advisory (Level 5), Warning (Level 6) and Crisis (Level 7). In an embodiment, the Artifact alarm events may be generated due to a poor quality alarm signal or patient intervention.

The alarm level adaptation module 106 is further configured to set an alarm rate limit that is associated with each alarm type. In an exemplary embodiment, the alarm rate limit is set based on population-average data and the identified pattern in the sequence of the alarm events and the alarm rate. In the illustrated embodiment, the alarm level adaptation module 106 compares the alarm rate of the alarm type with a pre-defined alarm rate limit. In an embodiment of the invention, the pre-defined alarm rate limit may be changed real-time based on the physiological condition of the patient. Accordingly, the alarm level adaptation module 106 adjusts the alarm level of each alarm type based on the pattern in the sequence of alarm events, the alarm rate and patient data. In an embodiment, the alarm level may be increased from a low level, say Advisory to a high level, say Crisis when the alarm rate or trend in alarm rate associated with the alarm type exceeds the pre-defined alarm rate limit. Similarly, when an exception is observed in the sequence of the alarm events, the alarm levels are adjusted. The patient data may include at least one of an age of the patient, gender of the patient, historical disease data associated with the patient, and historical alarm level data associated with the patient.

In various embodiments of the invention, the alarm levels are automatically modified from their default levels to indicate the degree of timeliness and criticality of service that is needed by the patient. The alarm level adaptation module 106 is further configured to suppress the alarm events and in turn the alarm signals based on one or more pre-defined conditions. In an embodiment, the one or more pre-defined conditions may include patient intervention or a pause signal generated by the hospital staff when the patients are being serviced. The alarm events are suppressed for a limited period of time until the patient intervention or the patient service is completed. In an embodiment, the alarm level adaptation module 106 allows the alarm signals to retain sensitivity to changes in the physiological monitoring system 100 during the service process. In other words, the alarm signals are logged but are not displayed when the patient service is in progress. Further, the alarm level adaptation module 106 restores the alarm level to a lower level or to it's base value (default setting).

The display panel 108 is configured to display the adjusted alarm level and the associated alarm type. The staff technician operating the physiological monitoring system 100 notifies the hospital staff based on the criticality of the alarm level displayed on the display panel 108. The staff technician may communicate with the hospital staff by using digital voice-over-internet-protocol (VoIP), telephony, paging capability or personnel tracking systems such as Radio Frequency Identification (RFID) badges. Thereafter, the hospital staff takes appropriate action based on the notification received. Examples of the display panel 108 may include, but are not limited to, a cathode ray tube (CRT) display, liquid crystal display (LCD) and a plasma display. In an embodiment, the display panel 108 may provide visual and auditory information regarding the adjusted alarm level and the associated alarm type.

Staff technicians at the physiological monitoring system 100 can also examine the current physiological condition of the patients at any time by directing the monitoring stations 102 to provide real-time patient data. Hence, in an event of occurrence of multiple alarm signals from multiple patients, the alarm levels of other monitoring stations may be reduced in such a way that only the most urgent alarms are generated. Thereafter, the staff technician may resolve the ongoing emergency situations.

In one embodiment of the invention, the physiological monitoring system 100 is a centralized patient monitoring system. It will be apparent to those skilled in the art that the physiological monitoring system 100 may also operate as a stand-alone device such as a bedside monitor.

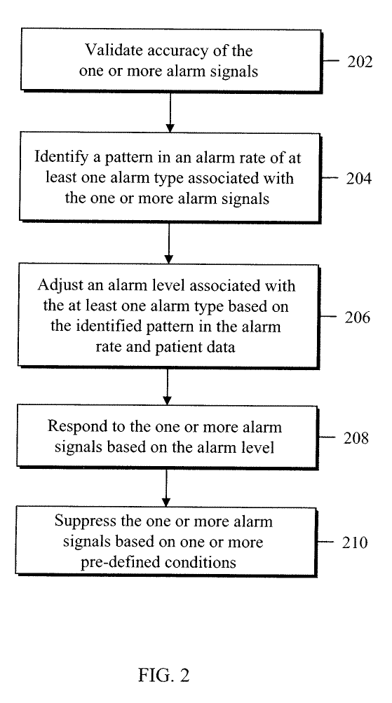

FIG. 2 is a flowchart illustrating a method for managing one or more alarm signals in the physiological monitoring system 100 in accordance with an exemplary embodiment of the invention. The alarm signals are associated with at least one alarm type. Examples of the alarm type include, but are not limited to, Premature Ventricular contraction (PVC), Asystole, Artifact, Heart Rate (HR), Couplet and Tachycardia. The alarm types indicate the physiological condition of the patients. At step 202, the processor 104 validates the occurrence of one or more alarm events associated with the one or more alarm signals corresponding to the one or more monitoring stations 102. An alarm event is detected when certain features are detected in the one or more alarm signals. In an embodiment, validating the alarm events may include checking (manually, or with a concurrent digital signal processor) if the one or more alarm events are noise. Further, it is also checked if the inter-alarm interval is typical or atypical for a patient having previously known medical conditions (part of the patient medical data). At step 204, the alarm level adaptation module 106 tracks the sequence of alarm events associated with the alarm type to determine a pattern or trend. Thereafter, at step 206, the alarm level adaptation module 106 adjusts an alarm level associated with the alarm type based on the identified pattern and patient data. For example, an alarm rate limit (the frequency of occurrence of the alarm event or alarm type) associated with the alarm type is set based on population-average data and the physiological condition of a patient. The population-average data includes data from a plurality of patients. Thereafter, at step 206, the alarm level adaptation module 106 adjusts an alarm level associated with the at least one alarm type based on the identified pattern in the sequence of the alarm events and patient data. For example, if the alarm rate for the alarm type is higher or lower than the set alarm rate limit the alarm level is adjusted. The patient data may include at least one of an age of the patient, gender of the patient, historical disease data of the patient, and historical alarm level data of the patient.

In this exemplary embodiment, the alarm rate is compared with the pre-defined alarm rate limit. The alarm level may be increased to a higher level to reflect the condition of the patient when a previous pattern in the alarm rate is validated and when the alarm rate exceeds the pre-defined alarm rate limit. The adjusted alarm level and the corresponding alarm type are displayed on the display panel 108. At step 208, the hospital staff responds to the one or more alarm signals based on the adjusted alarm level. At step 210, the alarm level adaptation module 106 suppresses the one or more alarm signals based on one or more pre-defined conditions. The one or more pre-defined conditions include at least one of patient intervention and a pause signal that is generated in the monitoring station 102 when the patient is being serviced. The physiological system resets the alarm levels, the alarm event tracker and the alarm rates when the alarm signals are suppressed.

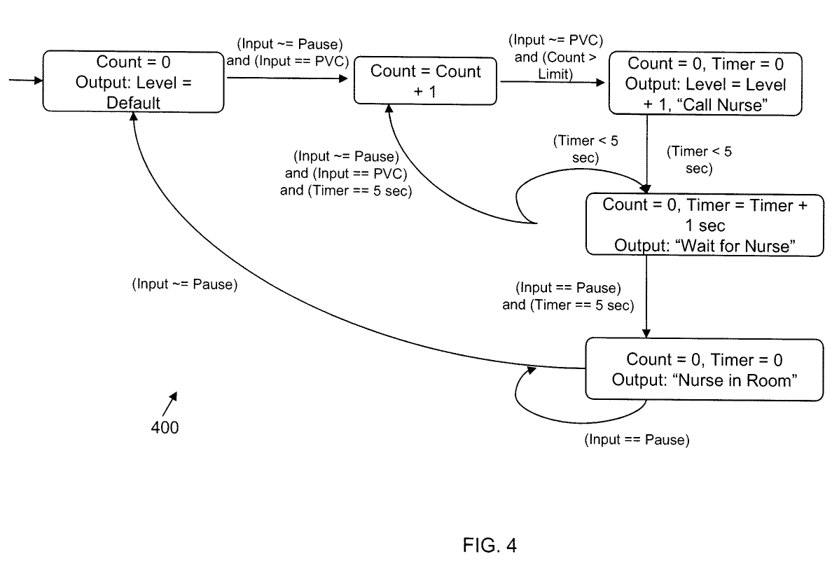

In an embodiment, each patient has a plurality of asynchronous state machines (timed automata), one for each alarm type, to maintain accurate alarm level control. The state logic for each state machine is designed to detect patterns in alarm timing associated with each phase of development of a patient condition (such as counting alarms within a time interval), escalation of the warning level (e.g., with increasing alarms per unit time), maintaining an alert until a staff response is indicated, and suppressing alarms during patient intervention. In this way, both short and long-term alarm patterns may be maintained, and all alarm levels can be adjusted concurrently. It will be apparent to a person skilled in the art that the alarm levels will be assigned to each alarm type at all times. It is the most recent alarm type, time and event that triggers the alarm level adaptation calculation. Hence over more extended periods of time, all alarm levels may drift gradually as the patient's condition changes. When no alarm events occur for extended periods of time, alarm level settings return to baseline values (either factory default values or those set by a particular hospital, typically). A PVC-level adapting state machine is explained in conjunction with FIG. 4.

Accordingly, the above described method shifts the alarm level of the alarm event (up or down) based on the pattern in the sequence of the alarm events, the physiological condition of the patient, and any detected or inferred patient interventions (such as “pause” events). Further, when an alarm event has a lower alarm level, it may be displayed as a text message on the screen of the monitoring stations 102 and when the alarm level is high, the alarm is accompanied by a klangon tone (horn or loud beeping). Herein, the term alarm represents the type of feature detected in the alarm signals, and the timed “event” of its detection by signal processing within the processor (102), or monitoring station (104).

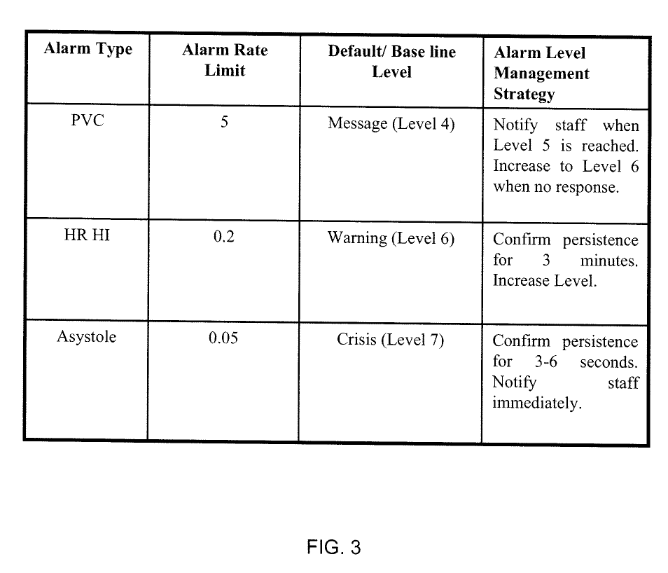

It is considered likely, due to its flexibility, that the method can be made compatible with existing equipment certification standards. In an embodiment, the method can obtain data from hospital information systems (HIS) to provide more precise timing of alarm level adaptation, and more customization to local clinical practices and treatment protocols. The above method is described with the help of examples described in FIG. 3.

FIG. 3 is a decision table illustrating an alarm level management strategy for various alarm types in accordance with an exemplary embodiment of the invention. In this embodiment, patient data from approximately 500 patients was previously analyzed to determine suitable alarm rate limits for each alarm type. The patient data includes age of the patient, gender of the patient, historical disease data of the patient and historical alarm level data of the patient. The alarm types considered here are Premature Ventricular contraction (PVC) alarm type, Heart Rate High (HR HI) alarm type and Asystole alarm type. As shown, the default alarm level for the PVC alarm type is set at Message (Level 4) and the alarm rate limit is set at 5 PVC alarm signals/ hour. Similarly, the default alarm level for the HR HI alarm type is set at Warning (Level 6) and the alarm rate limit is set at 0.2 HR HI alarm signals/hour. The default alarm level for Asystole alarm type is set at Crisis (Level 7) and the alarm rate limit is set at 0.05 Asystole alarm signals/ hour.

The physiological monitoring system 100 increments PVC alarm rate (on receipt of the PVC alarm signal) and compares the PVC alarm rate with the pre-defined PVC alarm rate limit (5 alarm signals/hour) when a PVC alarm signal is generated. In particular, alarm event sequence is checked to increase or decrease the alarm level. The physiological monitoring system 100 increases the alarm level from Message (Level 4) to Advisory (Level 5) when the pre-defined PVC alarm rate limit is exceeded. Accordingly, the staff technician operating the physiological monitoring system 100 notifies the hospital staff of the high PVC alarm rate and requests the hospital staff to take appropriate action. The alarm level is further increased to Warning (Level 6) when the hospital staff does not respond to the notification. The physiological monitoring system 100 expects a PAUSE signal from the monitoring station 102 associated with the patient when the hospital staff services the patient. The PVC alarm signal is suppressed, the PVC alarm rate is reset to zero and the PVC alarm level is reset to Message (Level 4) when the PAUSE signal is received.

Similarly, the physiological monitoring system 100 verifies if the received HR HI alarm signal is a part of a previous pattern. An HR HI alarm rate is incremented and compared with the pre-defined HR HI alarm rate limit (0.2 alarm events/hour) when the previous pattern is verified. The alarm level is raised to Crisis (Level 7) when the Warning (Level 6) persists for more than three minutes. Thereafter, the staff technician notifies the hospital staff and waits for the hospital staff to check the patient. The HR HI alarm signal is paused at the bedside while the hospital staff checks the patient. The hospital staff either adjusts medications for the patient or modifies the pre-defined HR HI alarm rate limit. The physiological monitoring system 100 suppresses the HR HI alarm signal, resets the HR HI alarm rate and resets the HR HI alarm level to Warning (Level 6) when either of these actions is taken.

Similarly, the physiological monitoring system 100 may confirm the persistence of the Asystole alarm signal for three to six seconds when the Asystole alarm signal is generated. A certification requirement sometimes requires the annunciation of critical alarm signals; for example, Asystole alarm signals must be sustained until they are manually cleared. The staff technician immediately summons the hospital staff and the patient is provided immediate attention.

FIG. 4 is a block diagram illustrating a PVC alarm state machine 400 in accordance with an exemplary embodiment of the invention. As illustrated in the figure, initially, the alarm level is set to zero (advisory). PVC alarm events are detected and a count is set for the detection of the alarm events. Based on the detection of PVC alarm events, the count is incremented. The count is compared with the PVC alarm count limit. If this condition persists, the alarm level is incremented to higher level, for example, Warning. Accordingly, the hospital staff is informed to visit the patient and review medication. Further, the alarm level adaptation methodology checks for receipt of a pause signal signifying the patient being serviced by the hospital staff, and decreases the alarm level accordingly. If the pause signal is not received within a pre-defined time interval (e.g. 5 seconds), the alarm level is further raised. If the pause signal is received with the pre-defined time interval, the alarm level is reset to zero and the count is also reset to zero.

In an embodiment, the method associated with each alarm type, as described herein, can be standardized using computer programming or engineering techniques including computer software, firmware, hardware or any combination or subset thereof. Hence, a limited amount of logic may be needed to implement the modulation of the alarm levels from a lower to higher level, or vice versa. Any such resulting program, having computer-readable code, may be embodied within one or more computer-readable media, thereby making a computer program product, i.e., an article of manufacture, according to the invention. The computer readable media may be, for instance, a hard drive, diskette, optical disk, magnetic tape, semiconductor memory such as read-only memory (ROM), etc., or any transmitting/receiving medium such as the Internet or other communication network or link. The article of manufacture containing the computer code may be made and/or used by executing the code directly from one medium, by copying the code from one medium to another medium, or by transmitting the code over a network.

One skilled in the art of computer science will easily be able to combine the software created as described with appropriate general purpose or special purpose computer hardware, such as a microprocessor, to create a computer system or computer sub-system embodying the method of the invention. An apparatus for making, using or selling the invention may be one or more processing systems including, but not limited to, a central processing unit (CPU), memory, storage devices, communication links and devices, servers, I/O devices, or any sub-components of one or more processing systems, including software, firmware, hardware or any combination or subset thereof, which embody the invention.

Various embodiments of the invention provide a method for managing one or more alarm signals in a physiological monitoring system that operates in a real-time monitoring environment. The physiological monitoring system adapts the alarm levels of the alarm types based on alarm certainty, phase of care, and patient medical data. . The adaptation logic includes associating the alarm levels of each alarm type with a previously identified pattern and determining the stage of patient intervention or patient service. The method uses standardized logic (concurrent finite state machines for each alarm type, driven by the detected alarm event string) to substantially reduce the number of alarm signals that are raised to higher levels during the service process, and to assure that this happens only when substantial confirming evidence is available. The method also allows the alarm levels of the alarm signals to be reduced when a pattern ceases on its own. Therefore, the method provides an advantage over the existing methods by being adaptive to individual patient conditions, hospital-specific service processes, patient demographics, or floor plans, and by reducing over-all auditory levels on the hospital ward.

The above described method and system has multiple advantages. The alarm level adaptation based on a previously identified trend or pattern, the level of urgency of response, and the stage of patient intervention helps in reducing alarm events that are raised to warning or crisis levels. Further, the method accommodates the patient intervention process by suppressing alarm levels during interventions. However, the adaptation logic also allows alarms to retain some sensitivity during service processes if this is appropriate (e.g., by setting the level to a low value, rather than “zero”).

While only certain features of the invention have been illustrated and described herein, many modifications and changes will occur to those skilled in the art. It is, therefore, to be understood that the appended claims are intended to cover all such modifications and changes as fall within the true spirit of the invention.

1. A method for managing one or more alarm events in a physiological monitoring system, the method comprising:
validating the one or more alarm events associated with one or more alarm signals;
identifying a pattern in the sequence of the one or more alarm events associated with at least one alarm type, the at least one alarm type being associated with the one or more alarm signals;
adjusting an alarm level associated with the at least one alarm type based on the identified pattern in the sequence of the one or more alarm events and patient data;
responding to the one or more alarm events based on the alarm level; and
suppressing the one or more alarm events based on one or more pre-defined conditions, wherein suppressing the one or more alarm events comprises automatically resetting the alarm level.

2. The method of claim 1 further comprising setting an alarm rate limit associated with the at least one alarm type based on population-average data and pattern in the alarm rate for a patient.

3. The method of claim 1 further comprising setting the alarm level based on the at least one alarm type and physiological condition of a patient.

4. The method of claim 1 further comprising checking if an alarm rate exceeds a pre-defined alarm rate limit.

5. The method of claim 1, wherein the patient data comprises at least one of an age of the patient, gender of the patient, historical disease data of the patient, and historical alarm level data for that patient.

6. The method of claim 1, wherein the one or more pre-defined conditions comprises at least one of patient intervention and a pause signal, the pause signal being generated when a patient is being serviced.

7. A physiological monitoring system comprising:
one or more monitoring stations configured to monitor one or more alarm events associated with one or more alarm signals, the one or more alarm signals being associated with one or more patients; and
a processor comprising an alarm level adaptation module configured to:
identify a pattern in sequence of detected alarm events associated with at least one alarm type by tracking an alarm rate associated with the alarm type for a pre-defined time interval, the at least one alarm type being associated with the one or more alarm signals;
set an alarm rate limit associated with the at least one alarm type based on population-average data and pattern in the alarm rate for a patient;
adjust the alarm level associated with the at least one alarm type based on the identified pattern in the sequence of the alarm events, the alarm rate and patient data; and
suppress the one or more alarm events via automatic resetting of the alarm level based on one or more pre-defined conditions.

8. The system of claim 7 further comprising a display panel configured to display the alarm level and the at least one alarm type.

9. The system of claim 7 further comprising a processor configured to validate one or more alarm events associated with the one or more alarm signals.

10. (canceled)

11. The system of claim 7, wherein the alarm level adaptation module is further configured to check if an alarm rate exceeds a pre-defined alarm rate limit.

12. The system of claim 7, wherein the alarm level adaptation module is further configured to set the alarm level based on the at least one alarm type and physiological condition of the one or more patients.

13. The system of claim 7, wherein the one or more pre-defined conditions comprises at least one of patient intervention and a pause signal, the pause signal being generated when the one or more patients are being serviced.

14. The system of claim 7, wherein the patient data comprises at least one of an age of the patient, a gender of the patient, historical disease data of the patient, and historical alarm level data for that patient.

15. The system of claim 7, wherein the physiological monitoring system is a centralized patient monitoring system.

16. The system of claim 7, wherein the one or more monitoring stations monitor one or more of an electrocardiogram (ECG), non-invasive blood pressure (NBP) and specific blood oxygen (SPO2).

17. The system of claim 7, wherein the alarm rate comprises a short-term average frequency of the occurrence of the one or more alarm events associated with an alarm type.

18. The system of claim 7, wherein the at least one alarm type comprises an alarm for premature ventricular contraction, asystole, artifact, heart rate, couplet and tachycardia.

19. The system of claim 7, wherein the alarm level comprises respective levels for a system message, a system advisory, a system warning, a message, an advisory warning and a crisis.
